(19) United States
(12) Patent Application Publication
(10) Pub. No.: US 2008/0241242 A1
(43) Pub. Date: Oct. 2, 2008

(54) Porous Polyelectrolyte Materials

(76) Inventors: Francesco Caruso, Victoria (AU); Yajun Wang, Victoria (AU)

Correspondence Address: HOVEY WILLIAMS LLP, 10801 Mastin Blvd., Suite 1000, Overland Park, KS, 66210, US

(21) Appl. No.: 11/576,681

(22) PCT Filed: Oct. 4, 2005
(86) PCT No.: PCT/AU05/01511

§ 371 Date: Jan. 14, 2008

(30) Foreign Application Priority Data
Oct. 5, 2004 (AU) 2004905787

(51) Int. Cl. A61K 9/00; B05D 5/00; B01J 19/00; A61P 43/00; B32B 5/22; B32B 9/04

(52) U.S. Cl. 424/484; 427/243; 205/334; 428/316.6; 428/315.5

(57) ABSTRACT

The present invention relates to porous polyelectrolyte materials, particularly nanoporous polyelectrolyte materials and to methods of making such materials. In a preferred embodiment, the invention relates to nanoporous polyelectrolyte spheres. In a preferred form of the invention, the materials are manufactured with the use of mesoporous silica spheres as templates. The invention also relates to a method of manufacturing such materials, and in particular, to a method of manufacturing such materials by a layer-by-layer process.

### FIELD OF THE INVENTION

The present invention relates to porous polyelectrolyte materials, particularly nanoporous polyelectrolyte materials and to methods of making such materials. In a preferred embodiment, the invention relates to nanoporous polyelectrolyte spheres. In a preferred form of the invention, the materials are manufactured with the use of mesoporous silica spheres as templates. The invention also relates to a method of manufacturing such materials, and in particular, to a method of manufacturing such materials by a layer-by-layer process.

### BACKGROUND OF THE INVENTION

Porous materials have been used as sacrificial host templates for the synthesis of various materials. In a typical synthetic strategy the constituent materials are infiltrated into the pores of the porous material and subjected to conditions such that reactions occur leading to the formation of an interconnected network within the pores. Removal of the template is then carried out to leave the final product. Mesoporous silicas are porous materials with extremely high surface areas and homogenous pores in the range of 2-50 nm. Mesoporous silicas may have a number of different shapes however in one known embodiment the mesoporous silica material is in the form of a particle or sphere. Silane grafting and in-situ synthesis doped with silane chemicals have been employed to modify the siliceous surface with various functional groups to tailor the functional properties of the material.

Due to the unique pore structure of mesoporous silica materials, these materials have been efficiently used as host porous supports or templates in replication synthesis. A mesoporous silica material, in particular, provides a confined space for controlled intra-pore inclusion of materials such as metals, metal oxides and carbons. These materials may be infiltrated into the pores, followed by reduction, crosslinking or carbonization to obtain an interconnected network, followed by removal of the silica template (typically by dissolution) to form a porous material. Porous materials of this type, especially in particulate form, are of interest in a diverse range of applications including controlled drug delivery, molecular separation technology and as hosts for chemical synthesis.

Accordingly it would be desirable to provide new materials of this type as well as new methods of making such materials, as it would be expected that the new materials may have a number of interesting properties.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

### SUMMARY OF THE INVENTION

The present invention aims to provide porous multilayer polyelectrolyte materials, particularly nanoporous multilayer polyelectrolyte materials. In one embodiment, the nanoporous polyelectrolyte material is substantially spherical and is produced by a layer-by-layer method utilizing a porous silica sphere, preferably a mesoporous silica sphere, as a template. Preparation of multilayer films has the benefit of low cost production, simplicity and versatility and has the potential for preparation of materials with designed morphologies in the presence of suitable templates.

Accordingly, in one embodiment of the invention, there is provided a porous multilayer polyelectrolyte material including at least two layers of polyelectrolyte material. In a particularly preferred embodiment the material includes at least two layers of oppositely charged polyelectrolyte material. In another preferred embodiment the material is a nanoporous multilayer polyelectrolyte material. The porous multilayer polyelectrolyte material is preferably spherical or substantially spherical.

The pores in the material may be of a wide variety of sizes however the material preferably includes pores with a pore size of from 5 to 50 nm, even more preferably 10 to 50 nm. In a particularly preferred embodiment the pores are interconnecting to produce an interconnected porous network.

The material may include any suitable number of polyelectrolyte layers with the number of layers being determined based on the desired properties of the final material produced. Nevertheless it is preferred that there are from two to ten layers of polyelectrolyte material, more preferably from two to eight layers of polyelectrolyte material, even more preferably from 4 to 8 layers of polyelectrolyte material. In one particularly preferred embodiment the material includes two layers of polyelectrolyte material. In one particularly preferred embodiment each layer of polyelectrolyte material is oppositely charged to the layer(s) of polyelectrolyte material adjacent to it. In another preferred embodiment the material includes at least two adjacent layers having the same charge. In a particularly preferred embodiment each layer of polyelectrolyte material is cross-linked. In one preferred form of the invention the cross-linking is such that one or more of the layers of polyelectrolyte material is cross linked to an adjacent layer. In another preferred form each layer is internally cross-linked. In a most preferred form of the invention the layers are both internally cross-linked and cross-linked to one or more adjacent layers.

The polyelectrolyte materials used to form the layers may be of any suitable polyelectrolyte material however it is preferred that each layer includes a polyelectrolyte material independently selected from the group consisting of polymers, biodegradable polymers, poly(amino acids), peptides, glycopeptides, polypeptides. peptidoglycans, glycosaminoglycans, glycolipids, lipopolysaccharides, proteins, glycoproteins, polycarbohydrates; polynucleotides, modified biopolymers; polysilanes, polysilanols, polyphosphazenes, polysulfazenes, polysulfide, polyphosphates, nucleic acid polymers, nucleotides, polynucleotides, RNA and DNA or a mixture thereof.

It is particularly preferred that each layer includes a polyelectrolyte material independently selected from the group consisting of polyglycolic acid (PGA), polylactic acid (PLA), poly-2-hydroxy butyrate (PHB), gelatins, (A, B) polycaprolactone (PCL), poly(lactic-co-glycolic acid) (PLGA), carboxymethyl cellulose, carboxymethyl dextran, poly(allylamine hydrochloride), poly(acrylic acid), poly(sodium 4-styrene sulphonate), poly(diallyldimethylammonium chloride), poly(vinylsulfate), poly(L-glutamic acid) and poly(L-lysine) or a mixture thereof.

It is particularly preferred that the polyelectrolyte material in at least one layer contains an amine group. In another preferred embodiment the polyelectrolyte material in at least one layer contains a carboxylic group. In a further preferred embodiment the material includes at least one layer of poly(acrylic acid). In another preferred embodiment the material includes at least one layer of poly(allylamine hydrochloride).

The molecular weight of the polyelectrolyte materials used to form the layers may vary widely with the molecular weights being chosen to provide the desired functionality to the finished product. It is preferred, however, that each polyelectrolyte material has a molecular weight of at least 100, more preferably at least 500. Accordingly each polyelectrolyte preferably has a molecular weight that is from 100 to 1,000,000, even more preferably from 500 to 1,000,000, even more preferably from 500, to 500,000, yet even more preferably 500 to 100,000, most preferably the polyelectrolyte material has a molecular weight of from 1000 to 100,000.

As stated above, the materials of the invention may incorporate a wide variety of polyelectrolyte materials depending upon the desired end use application of the porous multilayer polyelectrolyte material. As such one can select the surface or any of the layers of the material to impart the desired functionality on the material. In one preferred embodiment the material used to form at least one polyelectrolyte layer is selected from the group consisting of peptides, glycopeptides, polypeptides. peptidoglycans, glycosaminoglycans, glycolipids, lipopolysaccharides, proteins, glycoproteins and polynucleotides. In this embodiment it is preferred that at least one polyelectrolyte layer is a protein layer, preferably a protein layer wherein the protein has a molecular weight of from 1 to 500 kDa. In a particularly preferred embodiment the protein is selected from the group consisting of lysosome, cytochrome C, catalase, bovine serum albumin, immunoglobulin G, protease, RNase A, trypsin, conalbumin, lactoglobulin, myoglobin, ovalbumin, papain, penicillin acylase, and subtilisin Carlsberg.

As would be clear to a skilled addressee the polyelectrolyte materials of the various layers may be chosen to impart a wide variety of functionality on the end product.

The invention also relates to methods for the production of materials of this type which the applicants have found may be readily produced using the developed techniques.

In yet a further embodiment there is provided a method of manufacturing a porous multilayer polyelectrolyte material including the steps of:
(i) providing a porous template;
(ii) depositing layer-by-layer polyelectrolyte material onto the porous template; and
(iii) removing the template by exposure to a suitable solvent.

In one preferred embodiment the porous template is a mesoporous template, such as a mesoporous silica template. In this embodiment the polyelectrolyte material thus formed is a nanoporous material.

Any suitable template may be used in the method of the invention however it is preferred that the template has an interconnected network of pores. It is preferred that the template includes pores with a pore size in the range 2 to 50 nm, more preferably 10 to 50 nm. The template may be made of any suitable material but is preferably a silica template. The template may be any suitable shape but is preferably selected from the group consisting of planar supports, powder particles, fibres, tubes, films, membranes and spheres. A particularly preferred shape for the template is spherical or substantially spherical.

In certain embodiments of the method of the invention the exposed surface of the template is modified in order to facilitate bonding of the polyelectrolyte material to the template. In a particularly preferred embodiment the exposed surface has been modified by grafting 3-aminopropyltriethoxysilane (APTS) onto the exposed surface.

The steps of depositing the polyelectrolyte materials may be carried out in a number of ways but the polyelectrolyte material is preferably deposited in layers of alternating charge. In general the step of depositing the polyelectrolyte layers is carried out by contacting the template with a solution containing the polyelectrolyte material to be deposited. The solution may be of any suitable concentration but it will preferably have a concentration of polyelectrolyte material of 0.001 to 100 mg mL⁻¹, more preferably a concentration of polyelectrolyte material of 0.1 to 30 mg mL⁻¹, more preferably concentration of polyelectrolyte material of 0.5 to 10 mg mL⁻¹. In a particularly preferred embodiment the solution includes a salt. The salt preferably has a concentration of from 0.001 to 5 M, more preferably a concentration of from 0.05 to 5 M, most preferably a concentration of from 0.1 to 1 M. Any suitable salt may be used but it is preferred that the salt is sodium chloride.

The step of contacting the template with the solution may be carried out for any period of time suitable to achieve the desired deposition of polyelectrolyte. In one preferred embodiment the contacting is carried out for from 15 minutes to 24 hours, more preferably the contacting is carried out for from 2 hours to 20 hours, most preferably the contacting is carried out for from 4 hours to 12 hours.

In order to facilitate the deposition the solution is preferably subjected to ultrasound irradiation. It is preferred that following deposition each layer of polyelectrolyte material is cross-linked after being deposited and before deposition of a further layer. The cross-linking may be carried out using any technique well known in the art but is preferably cross-linked by heating at a temperature of from 100° C. to 250° C. or by other chemical means. In another preferred embodiment the polyelectrolyte layer is cross-linked using 1-ethyl-3-(3-dimethylaminopropyl) carbodiimide hydrochloride. The exact technique used to cross-link a layer will depend upon the chemical structure of the polyelectrolyte material used in that layer. Accordingly if cross-linking is desired this could readily be accomplished by a skilled addressee in the art.

In the methods of the invention the number of layers deposited may vary depending upon the desired end use application. In a preferred embodiment a plurality of layers are deposited. In one preferred embodiment two to ten layers are deposited, even more preferably from two to eight layers are deposited, most preferably from four to 8 layers are deposited.

The polyelectrolyte materials used in the methods of the invention may be chosen depending upon the desired end use application for the polyelectrolyte material to be manufactured. It is preferred that the polyelectrolyte material deposited to form each layer is independently selected from the group consisting of polymers, biodegradable polymers, poly(amino acids), peptides, glycopeptides, polypeptides. peptidoglycans, glycosaminoglycans, glycolipids, lipopolysaccharides, proteins, glycoproteins, polycarbohydrates, modified biopolymers; polysilanes, polysilanols, polyphosphazenes, polysulfazenes, polysulfide, polyphosphates, nucleic acid polymers, nucleotides, polynucleotides, RNA and DNA.

In a particularly preferred embodiment the polyelectrolyte material deposited to form each layer is independently selected from the group consisting of polyglycolic acid (PGA), polylactic acid (PLA), poly-2-hydroxy butyrate (PHB), gelatins, (A, B) polycaprolactone (PCL), poly(lactic-co-glycolic acid) (PLGA), carboxymethyl cellulose, carboxymethyl dextran, poly(allylamine hydrochloride), poly(acrylic acid), poly(sodium 4-styrene sulphonate), poly (diallyldimethylammonium chloride), poly(vinylsulfate), poly(L-glutamic acid) and poly(L-lysine) or a mixture thereof.

The molecular weight of the polyelectrolyte material used in the method of the invention may be chosen to provide the desired properties to the final product. It is preferred that each polyelectrolyte material has a molecular weight of at least 100, more preferably at least 500. Accordingly it is preferred that each polyelectrolyte material is chosen so that it has a molecular weight of 500 to 1,000,000, even more preferably each polyelectrolyte material has a molecular weight of from 500, to 500,000, yet even more preferably each polyelectrolyte material has a molecular weight of from 500 to 100,000, most preferably each polyelectrolyte material has a molecular weight of from 1000 to 100,000.

In one embodiment it is preferred that the polyelectrolyte material deposited to form at least one layer contains an amine group. In another embodiment the polyelectrolyte material deposited to form at least one layer contains a carboxylic group. In one particularly preferred embodiment the polyelectrolyte material deposited to form at least one layer is poly(acrylic acid). In another preferred embodiment the polyelectrolyte material deposited to form at least one layer is poly(allylamine hydrochloride).

The methods of the invention may be used to produce nanoporous biomaterials. In order to produce biomaterials of this type the polyelectrolyte material deposited to form at least one layer is selected from the group consisting of poly(amino acids), peptides, glycopeptides, polypeptides. peptidoglycans, glycosaminoglycans, glycolipids, lipopolysaccharides, proteins, glycoproteins, polycarbohydrates; nucleic acid polymers, nucleotides, polynucleotides, RNA and DNA, with proteins being particularly preferred. The protein may have any suitable molecular weight but preferably has a molecular weight of from 1 to 500 kDa, more preferably from 10 to 250 kDa. In a particularly preferred embodiment of the methods of the invention the protein is selected from the group consisting of lysosome, cytochrome C, catalase, bovine serum albumin, immunoglobulin G, protease, RNase A, trypsin, conalbumin, lactoglobulin, myoglobin, ovalbumin, papain, penicillin acylase, and subtilisin Carlsberg.

The removal of the template is preferably carried out by exposure to hydrofluoric acid. It is preferred that the hydrofluoric acid has a concentration of from 0.01 to 10 M, more preferably from 1 to 10 M, most preferably about 5 M.

In yet a further aspect the invention provides methods of delivering an active agent to a target site the method including the steps of (I) adsorbing the active agent onto a multilayer polyelectrolyte material of the invention and (ii) delivering the polyelectrolyte material to the target site. The active agent may be adsorbed in any of a number of ways but us typically adsorbed by suspending a polyelectrolyte material of the invention into a solution of the active agent. The active agent is adsorbed onto the polyelectrolyte material which can then be isolated from the solution. The polyelectrolyte material with the active agent adsorbed thereon may then be delivered to the target site such as by administration to the site so as to effectively deliver the active agent to the site. Any suitable active agent may be chosen for delivery such as therapeutic agents including pharmaceuticals, veterinary chemicals and the like. Alternatively the active agent may be a fragrance or a cleaning chemical which is intended to be delivered to its site of action. The target site may be any position or site that it would be desired for the active agent to be administered.

In a further aspect the present invention provides the use of a multilayer polyelectrolyte material of the invention as a micro reactor. It is found that the materials adsorb compounds and can thus be used to adsorb one or more reactive species allowing them to be held proximal to each other to facilitate reaction.

In yet an even further aspect the invention provides a method of conducting a chemical reaction including contacting a solution containing one or more reactants with a polyelectrolyte material of the invention. The step of contacting preferably involves addition of the polyelectrolyte material of the invention to a solution containing the reactant(s) in question. The chemical reaction may be carried out by the polyelectrolyte material acting as a micro reactor for the chemical reactant(s) as discussed above or the polyelectrolyte material may take an active part in the reaction. In a particularly preferred embodiment the reaction is an enzymatic reaction, preferably an enzymatic catalytic reaction of a reactant. In a most preferred embodiment the polyelectrolyte material catalyses the reaction.

As a result of their ability to adsorb chemical compounds the polyelectrolyte materials of the invention may be used as adsorbents. In yet an even further aspect the invention provides methods of removing a compound from solution including contacting the solution with a polyelectrolyte material of the invention, allowing sufficient time for the compound to be adsorbed by the polyelectrolyte material and removing the polyelectrolyte material from the solution. This method may be used to isolate drugs from solution or in the purification of solutions containing trace amounts of compounds that it is desired be removed from solution.

### DESCRIPTION OF THE FIGURES

FIG. 1 shows a schematic illustration showing the preparation of nanoporous polyelectrolyte spheres (NPS). 3-aminopropyltriethoxysilane (APTS)—modified spheres were layer-by-layer coated with polyelectrolytes of opposite charge [poly(acrylic acid) (PAA) and poly(allylamine hydrochloride) (PAH)] (steps 1 and 2, with the samples heated (160° C. for 2 h) after deposition of each polyelectrolyte to partially cross link the layers. The spheres were then dissolved by exposure to hydrofluoric acid (HF) (step 3) yielding intact NPS.

FIG. 2 (a) Fourier Transform Infrared (FTIR) spectra of the APTS-BMS spheres before and after the alternate deposition of PAA and PAH layers. The deposited layers were partially cross-linked by heating at 160° C. for 2 h prior to recording each spectrum. The numbers correspond to the number of polyelectrolyte layers deposited, commencing with PAA. APTS-BMS spheres were used as the internal reference for measuring each spectrum. The spectra are shifted in the vertical direction for clarity. (b) PAA amount deposited onto the APTS-BMS spheres as a function of PAA layer number, as determined by FTIR at 1720 cm⁻¹. The amount of PAA was calculated by using the absorbance of APTS-BMS at 800 cm⁻¹ as a reference and assuming an average APTS-BMS sphere size of 2.5 μm and a density of 0.53 g mL⁻¹.

FIG. 3 TEM images of the NPS comprised of (a) (PAA/PAH)₂/PAA (NPS-5) and ultramicrotomed thin sections of the same spheres at (b) low and (c) higher magnification. The NPS-5 was partially cross-linked by heating at 160° C. for 2 h after deposition of each polyelectrolyte layer. Images (b) and (c) clearly show the porosity of the polyelectrolyte spheres. The large difference in the diameters seen is a result of the ultramicrotoming process.

FIG. 4 SEM images of (a, b, c) NPS-5 [(PAA/PAH)₂/PAA] at different magnifications, and (d) (PAA/PAH)₂/PAA capsules prepared when PAA and PAH are deposited in the absence of added salt to the adsorption solution. The NPS-5 and capsules were partially cross-linked by heating at 160° C. for 2 h after deposition of each polyelectrolyte layer.

FIG. 5 CLSM images of FITC-labelled lysozyme immobilised in the NPS-5 [(PAA/PAH)₂/PAA] at (a) low and (b) higher magnification. The NPS-5 was partially cross-linked by heating at 160° C. for 2 h after deposition of each polyelectrolyte layer.

FIG. 6 Schematic illustration showing the preparation of nanoporous protein particles (NPP). Protein is first loaded in the mesoporous silica spheres (step 1), after which the protein molecules are bridged by the infiltrated polyelectrolyte (step 2). The mesoporous silica template is then dissolved by exposure to HF/NH₄F buffer (step 3), yielding intact NPP.

FIG. 7 Nitrogen sorption isotherms of the native MS spheres (diamonds), lysozyme-loaded MS (triangles), and the polyelectrolyte-connected protein in the MS after PAA infiltration and subsequent cross-linking (squares). (The open symbols in the nitrogen sorption isotherms correspond to the desorption branches.)

FIG. 8 TEM images of the NPP-lys (a) and NPP-cyt (b) prepared using 8,000 Da PAA as the bridging molecule. TEM images of NPP-lys prepared using PSS (70,000 Da) (c), and PAA (250,000 Da) (f) as the bridging molecules. SEM images (d, e) of the NPP-lys prepared using 8,000 Da PAA as the bridging molecule. The protein/PAA were cross-linked using EDC, followed by dissolution of the MS template using HF/NH₄F at pH ˜5.

FIG. 9 CLSM images of NPP-lys prepared using 8,000 Da (a) and 250,000 Da (b) PAA as the bridging molecules, respectively. Cross-linking of lysozyme/PAA was accomplished using EDC, after which the MS template was dissolved using HF/NH₄F at pH ˜5. The samples were incubated in 0.1 mg mL⁻¹ Rhodamine 6G solution for 60 min, washed with water four times, and then dispersed in 50 mM PB.

FIG. 10 TEM images of fibrous NPP-lys at different magnifications. PAA with molecular weight of 8,000 Da was used as the bridging molecule. Lysozyme/PAA was cross-linked using EDC as an initiator, followed by removal of the MS template with HF/NH₄F at pH ˜5.

### DETAILED DESCRIPTION OF THE INVENTION

As used herein the terms “polyelectrolyte” or “polyelectrolyte material” refers to a material that either has a plurality of charged moieties or has the ability to carry a plurality of charged moieties. A number of polyelectrolyte materials are well known in the art and the polyelectrolyte may be a positively charged polyelectrolyte (or have the ability to be positively charged) or a negatively charged polyelectrolyte (or have the ability to be negatively charged) or have a zero net charge. In addition the polyelectrolyte may be one where the charged moieties are relatively uniformly dispersed throughout the material (such as a charged polymer e.g. PAA) or may be one where the charged moieties are dispersed throughout the material. Proteins are an example of polyelectrolytes where the charged moieties are dispersed throughout the material as these molecules typically have areas of positive and negative charge dispersed throughout the molecule. As would be appreciated by a skilled worker, due to their ability to carry positive or negative charges, the term polyelectrolyte material therefore includes macromolecules which have the ability to carry a plurality of charges, including bio-macromolecules such as such as proteins, enzymes, polypeptides, peptides, polyoligonucleotides, polysaccharides, polynucleotides, DNA, RNA and the like.

### Porous Multilayer Polyelectrolyte Materials

As stated above the present invention provides a porous multilayer polyelectrolyte material including at least two layers of polyelectrolyte material. In a preferred embodiment the material includes at least two layers of oppositely charged polyelectrolyte material.

The layers of polyelectrolyte material may be attracted to each other via a number of mechanisms. Thus in one embodiment the layers are attracted via electrostatic interactions and thus it is the differential net charge of the adjacent layers that lead to the layers being held together. In this circumstance it is preferred that each layer is of alternating net charge such that each layer has an electrostatic attraction for the adjacent layer. In another embodiment the layers may be attracted to each other via hydrogen bonding interactions such that there is hydrogen bonding between the layers. Hydrogen bonding interactions may occur with layers of opposite net charge or of the same charge and thus provides a mechanisms whereby layers with the same net charge can be placed adjacent to each other.

Any suitable polyelectrolyte material may be used in each layer although as stated above it is preferred that each layer is of alternating charge such that each layer has an attraction for the adjacent layers. Examples of preferred polyelectrolyte materials for forming the layers include polymers, biodegradable polymers, poly(amino acids), peptides, glycopeptides, polypeptides. peptidoglycans, glycosaminoglycans, glycolipids, lipopolysaccharides, proteins, glycoproteins, polycarbohydrates, modified biopolymers; polysilanes, polysilanols, polyphosphazenes, polysulfazenes, polysulfide, polyphosphates, nucleic acid polymers, nucleotides, polynucleotides, RNA and DNA.

Examples of materials that can be used as polyelectrolyte materials include but are not limited to biodegradable polymers such as polyglycolic acid (PGA), polylactic acid (PLA), polyacrylic acid (PAA), polyamides, poly-2-hydroxy butyrate (PHB), gelatins, (A, B) polycaprolactone (PCL), poly(lactic-co-glycolic acid) (PLGA), flourescently labelled polymers, conducting polymers, liquid crystal polymers, photoconducting polymers, photochromic polymers; poly(amino acids) including peptides and S-layer proteins; peptides, glycopeptides, peptidoglycans, glycosaminoglycans, glycolipids, lipopolysaccharides, proteins, glycoproteins, polypeptides, polycarbohydrates such as dextrans, alginates, amyloses, pectins, glycogens, and chitins; polynucleotides such as DNA, RNA and oligonucleotides; modified biopolymers such as carboxymethyl cellulose, carboxymethyl dextran and lignin sulfonates; polysilanes, polysilanols, poly phosphazenes, polysulfazenes, polysulfide and polyphosphate. Preferred polymers include those with an amine group, for example poly(allylamine hydrochloride) or a carboxylic acid group, for example poly(acrylic acid). Other preferred polymers include poly(sodium 4-styrene sulphonate), poly(diallyldimethylammonium chloride), poly(vinylsulfate), et al., and the biocompatible polymers, such as poly(L-glutamic acid) and poly(L-lysine) and mixtures thereof.

Preferred polyelectrolyte materials include materials (including polymers) having a molecular weight of at least 100, more preferably at least 500. Accordingly it is preferred that the polyelectrolyte material is chosen so that the material has a molecular weight of from 100 to 1,000,000, more preferably from 500 to 1,000,000, more preferably from 500, to 500,000, even more preferably from 500 to 300,000, more preferably from 500 to 100,000, most preferably from 1000 to 100,000. Preferred polyelectrolyte materials include materials (such as polymers) with functional groups that can impart functionality on the porous material. For example it is preferred that the polyelectrolytes used in the process contain functional groups that can be cross-linked under suitable conditions to enable the various layers in the final material to be ultimately cross-linked.

In one particularly preferred embodiment the polyelectrolyte material in at least one layer contains an amine group. In another preferred embodiment the polyelectrolyte material in at least one layer contains a carboxylic group. It is particularly preferred that the material includes at least one layer of poly(acrylic acid). It is also particularly preferred that the material includes at least one layer of poly(allylamine hydrochloride).

In a particularly preferred embodiment at least one polyelectrolyte layer includes a material to impart a desired biological activity on the final polyelectrolyte material. This is preferably achieved by ensuring that at least one polyelectrolyte layer is selected from the group consisting of peptides, glycopeptides, polypeptides. peptidoglycans, glycosaminoglycans, glycolipids, lipopolysaccharides, proteins, glycoproteins and polynucleotides. If this is done it is preferred that the polyelectrolyte material is a protein, preferably a protein with a molecular weight of from 1 to 500 kDa. Examples of preferred proteins include of lysosome, cytochrome C, catalase, bovine serum albumin, immunoglobulin G, protease, RNase A, trypsin, conalbumin, lactoglobulin, myoglobin, ovalbumin, papain, penicillin acylase, and subtilisin Carlsberg.

The material included to impart a desired biological activity on the final polyelectrolyte material may be any layer of the final polyelectrolyte material. It may be an inner layer or it may be a surface layer.

The material should have at least two layers, but may in some circumstances have a far greater number of layers. A preferred configuration is from two to ten layers, more preferably from two to eight layers, most preferably from four to eight layers. In one particularly preferred embodiment the material has two layers of polyelectrolyte material. In one preferred embodiment each layer of polyelectrolyte material is oppositely charged to the layer(s) of polyelectrolyte material adjacent to it. It is found that this is advantageous as it facilitates attraction between the layers and provides a more stable final material. In an alternative embodiment the material includes at least two adjacent layers of polyelectrolyte material with the same net charge.

In many embodiments of the invention the layers of polyelectrolyte material are robust in that the attraction between molecules within a layer and between molecules of adjacent layers is such that the final multilayer polyelectrolyte material are relatively stable to a variety of conditions. In many instances these molecules are resistant to leaching such that there is no loss of polyelectrolyte from the molecule in solution. In some instances however the materials are not sufficiently stable to withstand the desired conditions of use and steps are preferably taken to increase the stability of the materials such as by cross-linking the materials.

In one preferred form, each layer of polyelectrolyte material is cross-linked in an intra-layer fashion. That is the layer is internally cross-linked such that molecules that make up the layer are linked to other molecules that make up the layer such that the layers consist of a web of cross-linked polyelectrolyte molecules.

In another preferred embodiment there is cross-linking between one or more adjacent layers of the multilayer polyelectrolyte material. In this embodiment molecules in one layer are linked to molecules in an adjacent layer. In this way networks of cross-linked molecules are created between adjacent layers. Of course as would be clear to a skilled addressee in the art a combination of cross-linking strategies may be employed such that the final multilayer polyelectrolyte material may have cross linking both within one or more layers and between one or more layers. It is found that cross-linking of this type strengthens the final material and increases its rigidity.

The material preferably has pores with a pore size of from 1 to 100 nm, more preferably 3 to 50 nm, even more preferably 5 to 50 nm, more preferably from 10 to 50 nm. As such the materials are preferably nanoporous materials. The pores are preferably interconnecting to produce an interconnected porous network. The material may be of any suitable shape but is preferably spherical.

The multilayer polyelectrolyte materials preferably have a particle size of from 0.1 to 1000 μm, more preferably from 0.1 to 100 μm, even more preferably 0.1 to 20 μm, most preferably from 0.4 to 5.0 μm. In one preferred embodiment the particles have a particle size of from 0.8 to 1.3 μm. In another preferred embodiment the particles have a size of from 1.4 to 2.1 μm. In yet another preferred embodiment the particles have a size of 1.6 to 2.4 μm.

The polyelectrolyte materials used in the layers of the multilayer polyelectrolyte material are preferably chosen such that the porous polyelectrolyte materials are self-supporting in that the pores do not collapse under the weight of the material after template removal. This may be achieved either by careful selection of the polyelectrolyte materials in the layers or by cross-linking of the layers to provide the required rigidity.

### Method of Production of the Materials of the Invention

As stated above the porous electrolyte materials of the invention are preferably produced using the layer-by-layer technique.

Accordingly the invention also provides a method of manufacturing a porous multilayer polyelectrolyte material including the steps of:
(iv) providing a porous template;
(v) depositing layer-by-layer polyelectrolyte material onto the porous template; and
(vi) removing the template by exposure to a suitable solvent.

The layer-by-layer technique typically exploits attractions between the respective layers to form the final multi-layer material. For example it may exploit the electrostatic attraction between oppositely charged species deposited from solution. Alternatively if the layers are held together by hydrogen bonding interactions it typically exploits the hydrogen bonding interactions between the polyelectrolyte materials chosen. In one preferred embodiment the polyelectrolyte material will be deposited in alternating positive and negative layers. In another preferred embodiment the polyelectrolyte material is deposited such that there are at least two adjacent layers with the same net charge. The subsequent removal of the template leaves a porous polyelectrolyte material, preferably a nanoporous polyelectrolyte material.

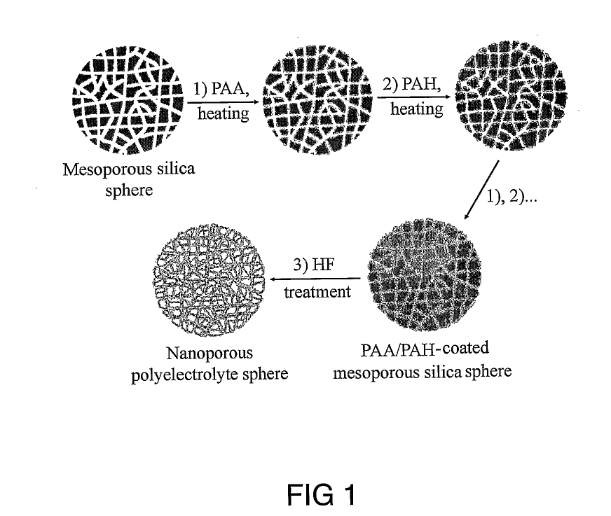

FIG. 1 shows a schematic depiction of the preparation of a preferred embodiment, namely a nanoporous polyelectrolyte sphere utilising PAA and PAH. As depicted the process involves using a mesoporous silica sphere and depositing a layer of PAA (step 1). This is followed by deposition of a layer of PAH (step 2) and then steps 1. and 2 are repeated the desired number of times depending upon the number of layers desired in the final material. Once the desired number of layers has been deposited the process includes treatment of the material to remove the template (step 3) to form the final porous multilayer polyelectrolyte material.

The porous template may be of any suitable type that provides an interconnected network of pores. The pores may take a number of different shapes and sizes however it is preferred that the porous template is a mesoporous template. Mesoporous templates are templates in which there are at least some pores, preferably a majority of pores having a pore size in the range 2 to 50 nm. The mesoporous template may be made of a number of suitable materials that allow for their subsequent removal although the template is preferably a mesoporous silica material. The template may take any suitable form and may be for example in the form of planar supports, powder particles, fibres, films, membranes or spheres. It is preferred that the template is spherical or substantially spherical.

It is most preferred that the mesoporous material is a mesoporous silica sphere in order to produce a spherical or substantially spherical nanoporous polyelectrolyte material. It will be convenient to describe the invention in terms of a spherical material, but it shall be kept in mind that the porous polyelectrolyte material produced by the process of the invention may be of any form, depending on the form of the template. Thus in general the final shape of the porous polyelectrolyte materials produced by the process of the invention will take the general shape or form of the template used in their synthesis. Thus for example if the template is spherical then the final product will typically be spherical. If the template is a fibre then once again the final product will typically be a fibre.

With many polyelectrolytes and templates the attraction between the polyelectrolyte and the surface of the pores of the template is such that the polyelectrolyte is naturally adsorbed onto the surface of the pores of the template. In such cases the template can be used directly in the process without any modification. In certain circumstances, however, the surface of the pores of the template and the polyelectrolyte may not have sufficient affinity for the polyelectrolyte to be efficiently adsorbed onto the surface of the pores. In these cases it is preferred to modify the exposed surface of the pores of the template prior to depositing the polyelectrolyte.

The surface of the pores of the template may be modified by addition of functional moieties to enhance the adsorption of the polyelectrolyte onto the pore surface. Any of a number of functional moieties can be added onto the surface of the pores of the template with the choice of functional moiety being chosen to complement the polyelectrolyte being introduced as the first layer during the process. A skilled worker in the area will generally have little difficulty in choosing a functional moiety to introduce onto the surface of the template to complement the chosen polyelectrolyte. A particularly preferred method of modifying the surface of a silica template for example is to graft a moiety such as 3-aminopropyltriethoxysilane (APTS) onto the surface of the silica. This introduces an amine surface functionality that can react with any carboxyl groups on the polyelectrolyte to promote adsorption of the electrolyte. If it was desired to promote adsorption of a polyelectrolyte that contains amino moieties this could similarly be carried out by attaching carboxyl moieties to the exposed surface of the template.

As stated above it is preferred that the porous template is a mesoporous silica material. In general, the mesoporous silica material may have a bimodal pore structure, that is, having smaller pores of about 2-3 nm and larger pores from about 10-40 nm.

The layers of polyelectrolyte material may be deposited in any of a number of orders and the order of deposition of the layers will depend upon the desired final layer order in the final multilayer polyelectrolyte material. In one embodiment it is preferred that the polyelectrolyte layers are deposited in layers of alternating charge. In another preferred embodiment layers of the same net charge may be deposited one after the other. As would be appreciated by a skilled addressee a combination of these two embodiments may also be used.

Each layer of polyelectrolyte material is typically deposited onto the porous template by contacting the porous template with the polyelectrolyte. The contacting of the porous template with polyelectrolyte typically involves the polyelectrolyte material being applied to the template such as a mesoporous template in solution form in a suitable solvent. Generally, the polyelectrolyte material when applied in solution, will be in the form of an aqueous solution, preferably an aqueous salt solution. The polyelectrolyte in solution typically has a concentration of from 0.001 to 100 mg mL⁻¹, more preferably from about 0.1 to 30 mg mL⁻¹, most preferably from 0.5 to 10 mg mL⁻¹.

If a salt solution is used the salt in solution preferably has a concentration of from about 0.001 to 5 M, more preferably from 0.05 to 5 M, most preferably from 0.1 to 1 M. It is preferred that the polyelectrolyte material is applied in a salt solution as, without wishing to be bound by theory, it is thought that in the presence of a salt solution, the polyelectrolyte material can be highly coiled which assists in it penetrating into the mesopores of the template. In the absence of salt, the polyelectrolyte will be mainly restricted to the outer surface assembly of the template as it will be presented with a long chain configuration. The salt may be of any suitable type but is typically selected from the group consisting of potassium chloride, lithium chloride and sodium chloride with sodium chloride being particularly preferred.

The polyelectrolyte material will generally infiltrate the pores within the range of 10-40 nm. Preferred polyelectrolyte materials include any known polymer material having a molecular weight of at least 100, more preferably from 500 to 1,000,000, more preferably from 100 to 500,000, even more preferably from 500 to 100,000, most preferably from 1000 to 100,000. Preferred polyelectrolyte materials include polymers with functional groups that can impart functionality on the porous material. For example it is preferred that the polyelectrolytes used in the process contain functional groups that can be cross-linked under suitable conditions to enable the various layers in the final material to be ultimately cross-linked.

Any suitable polyelectrolyte material may be used in each layer although it is preferred that each layer is of alternating charge such that each layer has an attraction for the adjacent layers. Examples of preferred polyelectrolyte materials for forming the layers include polymers, biodegradable polymers, poly(amino acids), peptides, glycopeptides, polypeptides. peptidoglycans, glycosaminoglycans, glycolipids, lipopolysaccharides, proteins, glycoproteins, polycarbohydrates, modified biopolymers; polysilanes, polysilanols, polyphosphazenes, polysulfazenes, polysulfide, polyphosphates, nucleic acid polymers, nucleotides, polynucleotides, RNA and DNA.

Examples of materials that can be used as polyelectrolyte materials include but are not limited to biodegradable polymers such as polyglycolic acid (PGA), polylactic acid (PLA), polyacrylic acid (PAA), polyamides, poly-2-hydroxy butyrate (PHB), gelatins, (A, B) polycaprolactone (PCL), poly(lactic-co-glycolic acid) (PLGA), flourescently labelled polymers, conducting polymers, liquid crystal polymers, photoconducting polymers, photochromic polymers; poly(amino acids) including peptides and S-layer proteins; peptides, glycopeptides, peptidoglycans, glycosaminoglycans, glycolipids, lipopolysaccharides, proteins, glycoproteins, polypeptides, polycarbohydrates such as dextrans, alginates, amyloses, pectins, glycogens, and chitins; polynucleotides such as DNA, RNA and oligonucleotides; modified biopolymers such as carboxymethyl cellulose, carboxymethyl dextran and lignin sulfonates; polysilanes, polysilanols, poly phosphazenes, polysulfazenes, polysulfide and polyphosphate Preferred polymers include those with an amine group, for example poly(allylamine hydrochloride) or a carboxylic acid group, for example poly(acrylic acid). Other preferred polymers include poly(sodium 4-styrene sulphonate), poly(diallyldimethylammonium chloride), poly(vinylsulfate), et al., and the biocompatible polymers, such as poly(L-glutamic acid) and poly(L-lysine) and mixtures thereof.

It is particularly preferred that at least one polyelectrolyte layer is selected from the group consisting of poly(amino acids), peptides, glycopeptides, polypeptides. peptidoglycans, glycosaminoglycans, glycolipids, lipopolysaccharides, proteins, glycoproteins, polycarbohydrates; nucleic acid polymers, nucleotides, polynucleotides, RNA and DNA. If this is done it is preferred that the polyelectrolyte material is a protein, preferably a protein with a molecular weight of from 1 to 500 kDa. Examples of preferred proteins include lysosome, cytochrome C, catalase, bovine serum albumin, immunoglobulin G, protease, RNase A, trypsin, conalbumin, lactoglobulin, myoglobin, ovalbumin, papain, penicillin acylase, and subtilisin Carlsberg.

After addition of the solution of the polyelectrolyte material to the template the mixture thus formed is typically agitated to allow the polyelectrolyte material to be adsorbed into the pores of the template. This can be done for any suitable length of time but it is typically found that the solution is agitated from 15 minutes to 24 hours, more preferably from 2 hours to 20 hours, even more preferably from 4 hours to 12 hours, most preferably about 6 hours.

Preferably ultrasound may be used during the depositing of the polyelectrolyte on the mesoporous template to assist in allowing the polyelectrolyte material to infiltrate the pores of the mesoporous template. Accordingly after the solution of polyelectrolyte material has been mixed with the template the mixture may be ultrasonicated. It has been found that with the application of ultrasound, together with agitation of the mixture of polyelectrolyte material and the mesoporous template, that higher molecular weight polymer material can infiltrate the pores in an efficient manner.

Following the mixing as discussed above the template may be treated to remove excess polyelectrolyte material that has not been adsorbed onto the template. The treatment may involve centrifugation, washing or a mixture thereof. This removes excess solution and increases the prospect that the next layer will be able to be successfully added.

In a preferred process for the preparation of the nanoporous polyelectrolyte material, the layers of polyelectrolyte are deposited layer-by-layer with oppositely charged polyelectrolyte materials. That is, there will be a build-up of subsequent positive and negatively charged materials. The material should have at least two layers, but may in some circumstances have a far greater number of layers. In principle the only limitation on the number of layers is the pore size of the porous template. Eventually as a plurality of layers are laid down they fill the pores completely thus stopping infiltration of any further polyelectrolyte material. A preferred configuration is from two to ten layers, more preferably from four to eight layers.

In one preferred form, each layer of polyelectrolyte material is cross-linked internally after being deposited and before deposition of a further layer. The layer may be cross-linked in any way well known in the art with the method chosen being determined by the moiety on the polyelectrolyte material that allows cross-linking.

In one preferred embodiment the polyelectrolyte layers are cross-linked by subjecting them to heat. The cross-linking is generally performed by heating at a temperature of from about 100° C. to 250° C., more preferably from 140° C. to 220° C., most preferably about 160° C. The amount of time taken to effect cross-linking will vary depending on the nature of the cross-linking moieties but it typically takes from 30 minutes to 12 hours.

In another preferred embodiment the layer may be internally cross-linked using other chemical means such as by the use of 1-ethyl-3-(3-dimethylaminopropyl)carbodiimide hydrochloride. The exact means of chemical cross-linking of the materials will depend upon the nature of the polyelectrolyte materials chosen.

Using similar methodology the layers may also be cross-linked to each other to provide further strength to the final multilayer material formed.

Cross-linking the polyelectrolyte material reinforces the strength of the polyelectrolyte layer. The layers may also be cross linked in an inter layer fashion such that one layer is cross-linked to the adjacent layers. This is generally carried out by reaction of the layers with a chemical entity that is able to react with functional groups on each layer.

In an alternative embodiment it is sometimes possible to cross-link the layers after deposition of all layers of polyelectrolyte material.

Following the deposition of the desired number of layers the process then involves removal of the template. The template may be removed by exposure to a suitable solvent that is capable of dissolving the template. In general the solvent will be chosen such that it is able to dissolve the template but such that it will not damage the polyelectrolyte layers. An example of a suitable solvent is hydrofluoric acid or sodium hydroxide. It has been found that the silicone dioxide core of the mesoporous silica material can readily be decomposed in hydrofluoric acid as it is converted to [SiF₆]²⁻ ion leaving the polyelectrolyte layers. Preferably, the mixture containing the template is shaken when the template is exposed to the hydrofluoric acid. If hydrofluoric acid is used it is found that the silica can be dissolved using a wide range of concentrations of acid. The acid may be of any strength although it is convenient to use an acid strength of from 1 to 10 M, more preferably about 5 M. Whereas hydrofluoric acid is preferred as a solvent, other suitable solvents would be well appreciated by the skilled practitioner. As such in principle any substance that can dissolve the template may be used as the solvent.

The main advantages of the layer-by-layer approach in the formation of a nanoporous polyelectrolyte sphere is that it offers a facile route to nanoporous polyelectrolyte sphere production as it is based on self assembly of a multilayer species based on attractions between the layers such as electrostatic self-assembly principles, thereby allowing the preparation of nanoporous polyelectrolyte spheres of diverse composition. Further, it affords nanometer level control of the deposited polyelectrolyte thickness and hence allowing the control of the functional groups of the mesoporous silica materials and subsequently on the nanoporous polyelectrolyte sphere, depending on the number of layers deposited.

Using the bimodal mesoporous silica spheres as a template has at least the following benefits:
(i) relatively regular spherical morphology makes it easier and possible to follow the particle morphology variation after polyelectrolyte deposition (it is difficult to do so with mesoporous silica with fractal morphology);
(ii) bimodal mesoporous silica possesses large mesopores (10-40 nm) and very high pore volume (1.2 mL g⁻¹) for such pores, which provides comparable size for the polyelectrolyte layer-by-layer infiltration into the three-dimensional random pores in the bimodal mesoporous silica templates. Self-standing nanoporous polyelectrolyte spheres are yielded after removal of the bimodal mesoporous silica templates.

Control of the functional groups within the siliceous mesopores can also improve the material performance in the applications for which the nanoporous polyelectrolyte sphere may be used. For example, depending on the functional group of the nanoporous polyelectrolyte sphere, the material may find use in bio-molecule (i.e. protein) adsorption/separation, high efficient adsorbents for environmental protection (i.e. removal of heavy metal ions and toxic organic molecules), enzyme immobilization and drug delivery. In addition the final materials may be useful as adsorbents for dyes and could also be useful in the controlled release of fragrances in certain applications.

The porous polyelectrolyte materials of the invention may also be coated using the layer-by-layer technique discussed herein to produce a shell on the outer surface assembly of the porous polyelectrolyte material. This may be useful in some applications such as where one desires to encapsulate any entities adsorbed into the pores of the porous polyelectrolyte material. Accordingly, one could treat the porous polyelectrolyte materials of the invention in solution to adsorb a material of interest such as an enzyme or pharmaceutically active compound. Once the desired amount of material had been absorbed into the pores, the porous polyelectrolyte material could then be surface coated using the methodology discussed above to produce an encapsulated material. This could be used in applications such as sustained drug delivery or the like by judicious selection of the coating layers.

The invention will now be described with reference to the accompanying examples.

### EXAMPLES

Materials: Catalase (C-100), cytochrome C (C-2037), poly(acrylic acid) (PAA, M_{w} 8,000, and 250,000), poly(sodium 4-styrenesulfonate (PSS, M_{w} 70,000), poly(L-glutamic acid) (PGA, M_{W} 1,500-3,000), 1-ethyl-3-(3-dimethylaminopropyl)carbodiimide hydrochloride (EDC), hydrogen peroxide (H₂O₂), hydrofluoric acid (HF), ammonium fluoride (NH₄F), sodium metasilicate (Na₂SiO₃) and cetyltrimethylammonium bromide (CTABr) were obtained from Sigma-Aldrich and used as received. Lysozyme was purchased from Fluka BioChemika. The mesoporous silica (MS) spheres were synthesised according to a literature method (G. Schulz-Ekloff, J. Rathouský, A. Zukal, Int. J. Inorg. Mater. 1999, 1, 97). All PE solutions were of concentration 5 mg mL⁻¹. The solution used for dissolving the silica core was a mixture of 2 M HF and 8 M NH₄F at pH ˜5. The water used in all experiments was prepared in a Millipore Milli-Q purification system and had a resistivity higher than 18 MΩ cm.

### Example 1

### Production of Nanoporous Polyelectrolyte Spheres (NPS) Using Poly(Acrylic Acid) (PAA) and Poly(Allylamine Hydrochloride) (PAH)

A mesoporous silica sphere was prepared in accordance with the method described in a paper published in the International Journal of Inorganic Materials (1999) 97-102, titled Mesoporous Silica with Controlled Porous Structure and Regular Morphology by Schulz-Ekloff et al. The molar ratio of surfactant to silica (CTABr/Na₂SiO₃) used to prepare the material is much higher (ca. two times) than that used to prepare conventional mesoporous materials. Therefore, the produced particles contain domains with stable silica walls between the micelles as well as domains in which these walls are unstable or even missing, which will form the larger mesopores after the removal of surfactant micelles. The mesoporous silica sphere possesses a bimodal mesoporous silica structure. That is, the bimodal mesoporous silica template has a surface area of 630 m²g⁻¹ and a pore volume of 1.72 mL g⁻¹. The material has a bimodal pore structure that is smaller pores in the range of 2-3 nm and larger pores in the range of from 10-40 nm with a volume of 1.28 mL g⁻¹. The bimodal mesoporous silica spheres have a particle size distribution of 2-4 μm.

To modify the bimodal mesoporous silica surface with a layer of functional groups (e.g. —NH₂ groups), which will have specific adsorption with subsequent polyelectrolyte deposition, a silanization method is applied according to the literature method. In this process, the newly dried mesoporous silica powder was well dispersed in toluene by sonication for 20 min before silane chemicals were added to the suspension. The molar ratio of the mesoporous silica particles (calculated as SiO₂)/silane chemical/toluene was fixed to be 5:1:500, and the suspension was refluxed for 24 h. The silane grafted mesoporous silica particles were separated from the solution by centrifugation, and washing in toluene and methanol twice, respectively. Finally, the pellet was dried at 80° C. for 12 h. The silane modification was fulfilled through grafting 3-aminopropyltriethoxysilane (APTS) on the bimodal mesoporous silica pore walls. The APTS grafted bimodal mesoporous silica (denoted as APTS-BMS) has a surface area of 465 m²g⁻¹, and a pore volume of 1.32 mL g⁻¹. Most of the pore volume in the APTS-BMS is contributed by the mesopores ranging from 10-40 nm with a volume of about 1.0 mL g⁻¹.

Poly(acrylic acid) (PAA) having a molecular weight of 2000 and poly (allylamine hydrochloride) (PAH) having a molecular weight of 15,000 were used as the counter polyelectrolyte pairs for the layer-by-layer assembly in the mesoporous silica spheres. All polyelectrolyte solutions were of a concentration of 5 mg mL⁻¹ and contained 0.7 M NaCl. The adsorption of polyelectrolyte was processed at ambient temperature for fifteen minutes in a sonication bath, followed by shaking for six hours. The sample was separated following three minutes by centrifugation (500 g) and washed with 0.1M NaCl solution four times.

To reinforce the polyelectrolyte on the walls, cross-linking of the polyelectrolyte after each layer of polyelectrolyte deposition was applied. The cross-linking was performed by heating of the sample at 160° C. for two hours, according to a method described in the International Journal of Journal of the American Chemical Society (1999) 1978-1979, titled Synthesis of Passivating, Nylon-Like Coatings through Cross-Linking of Ultrathin Polyelectrolyte Films by Jeremy J. Harris et al. Under this treatment it was found that amide bonds are formed by the —COOH groups (in PAA) and the —NH₂ groups (in PAH).

It has been found that the polyelectrolytes assembled in the BMS particles in the subsequent polyelectrolyte assembly step may dissolve and may form aggregates on the particle surface and solution if the materials are prepared without cross-linking. If cross-linking is applied, the surface of the polyelectrolyte assembled APTS-BMS particles is very smooth and the pore structure still can be distinguished by transmission electron microscopy (TEM) at high magnification in the APTS-BMS-polyelectrolyte samples and indicate negligible aggregation on the APTS-BMS surface. This result means that cross-linking can effectively stabilise the polyelectrolyte layers, and avoid desorption of the previous polyelectrolyte layer in the subsequent polyelectrolyte assembly.

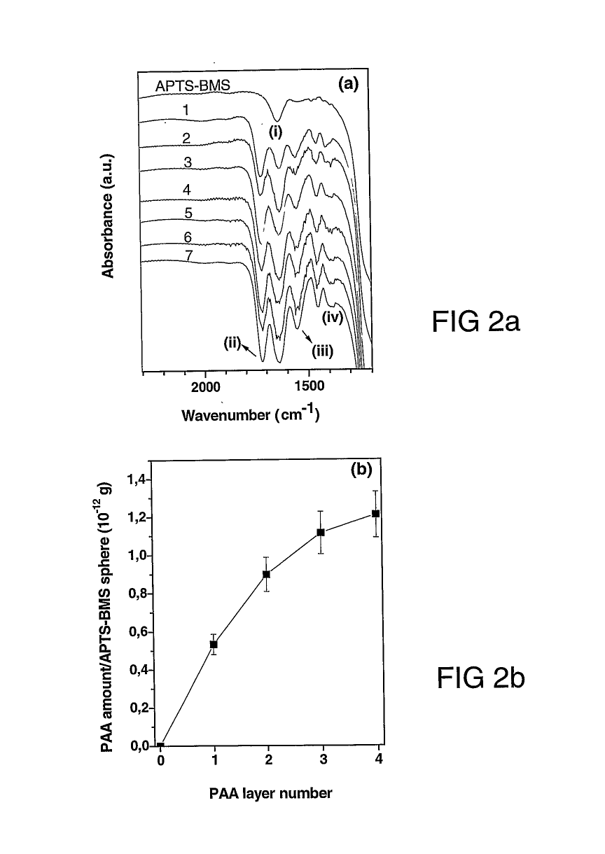

The successful deposition of the PAA and PAH in the APTS-BMS particles is further forcefully proved by FTIR. The FTIR spectra of the APTS-BMS particles after different layer of PAA and PAH deposition are shown in FIG. 2(a). For the APTS-BMS spheres, the absorption band at 1635 cm⁻¹ (i) is assigned to the Si—OH vibrations and the N—H bending (scissoring) vibrations of APTS. The peaks at 1720 (ii), 1570 (iii) and 1400 (iv) cm⁻¹ are attributed to the —COOH carbonyl and —COO⁻ asymmetric and symmetric stretches, respectively, of PAA. The intensities of the peaks at 1635 cm⁻¹ (due to the N—H bending (scissoring) vibration of PAH for layer number ≧2) and at 1720 cm⁻¹ (corresponding to PAA deposition) increase with PAH and PAA layer number, respectively, confirming the sequential deposition of PAA/PAH multilayers. The following observations can be made from the spectra: (a) The presence of —COOH (from PAA) after heating at 160° C. indicates that only partial cross-linking of the layers occurs. Only ca. 10-15% reduction in intensity of this peak was observed after heating the films. (b) The amide bonds formed as a result of cross-linking (peak at ˜1670 cm⁻¹) are not discernible, largely due to the relatively low cross-linking degree and masking from the peak at 1635 cm⁻¹ (arising from the APTS-BMS substrate and PAH). (c) The total amount of PAA deposited per APTS-BMS particle increases with PAA layer number, although the amount adsorbed per layer decreases with increasing PAA layer number (FIG. 2b). This trend is attributed to increased blockage of the larger mesopores in the APTS-BMS templates with increasing polyelectrolyte layer number.

The PAA deposition amount via the layer numbers is depicted in FIG. 2b. With the first layer of PAA deposition, the sample weight (i.e. PAA deposition amount) increased about 14 wt % of the original APTS-BMS templates. After that, the deposition amount in each layer will gradually decrease with the layer numbers increasing, which might be caused by partially blocking of the smaller mesopores in the APTS-BMS templates.

To examine the influence of sphere porosity, the experiment used both mesoporous silica spheres with only 2-3 nm pores and nonporous silica spheres for comparison. No distinguishable peaks due to PAA and PAH in the FTIR spectra of either of the PAA/PAH-coated silica spheres were observed, even after deposition of seven layers (i.e., 3.5 PAA/PAH bilayers). This indicates that polyelectrolyte deposition predominantly occurs in the larger mesopores of the APTS-BMS particles, and that the contribution to the FTIR intensities from polyelectrolyte adsorption on the outer surface of the particles is negligible.

The silicon dioxide skeleton was dissolved by hydrofluoric acid (10% solution in water), while gently shaking the tubes for twelve hours. The silicon dioxide core can be decomposed in 1 M hydrofluoric acid within a few seconds into [SiF₆]²⁻ ion, which can leave the polyelectrolyte layers during the dissolution without problems. Effective removal of the silicon dioxide wall is proved by energy dispersive X-ray and FTIR spectra. Only a small amount of silicon (0.8%) was detected after the template removal. The small amount of silicon is most possibly caused by the silicon-alkyl groups (arising from APTS modification), which is stable in the presence of hydrofluoric acid.

Nitrogen adsorption measurements were also conducted to follow the changes in the surface area of the APTS-BMS spheres after polyelectrolyte deposition. The first layer of adsorbed PAA dramatically decreased the surface area from 465 m²g⁻¹ (APTS-BMS template) to 284 m²g⁻¹. This is likely caused by the high PAA loading and blocking of some of the mesopores. Deposition of subsequent PAH and PAA layers resulted in a surface area decrease per polyelectrolyte adsorption step of approximately 20 m²g⁻¹. After deposition of seven layers, the surface area of the coated spheres was ca. 160 m²g⁻¹. These data further confirm the stepwise deposition of polyelectrolytes within the APTS-BMS spheres.

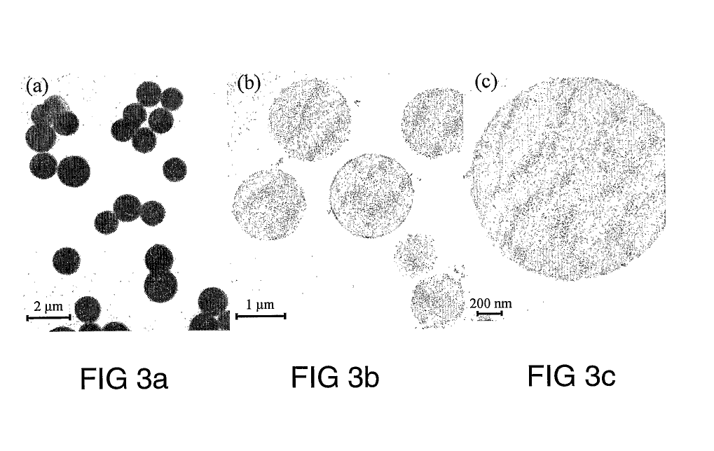

TEM was used to follow the nanoporous polyelectrolyte sphere morphology and size with the layer number variation. The nanoporous polyelectrolyte sphere prepared with different layer numbers of polyelectrolyte is denoted as NPS-n. It was found that the nanoporous polyelectrolyte materials of the invention typically retain the original shape of the template and do not show any signs of collapse. The APTS-BMS-PAA sample (one layer of PAA deposition) totally dissolved in seconds after exposure to hydrofluoric acid solution. Spherical morphology can be obtained for the sample (i.e. NPS-2 sample) with an additional layer of PAH deposition on the APTS-BMS-PAA sample. The NPS-2 sample had a diameter of from 0.8 to 1.3 μm, representing shrinkage of about 55% compared to the original APTS-BMS templates. With more layers of polyelectrolyte, less shrinkage was found in the nanoporous polyelectrolyte sphere products. For the NPS-7 sample, shrinkage of about 25% was found after the silica skeleton dissolution (diameter of approximately 1.4 to 2.1 μm). No obvious aggregation of the nanoporous polyelectrolyte sphere was found from TEM low magnification images (FIG. 3a). The inner structure of the nanoporous polyelectrolyte sphere was examined by slicing the spheres to a thickness of about 90 nm using the TEM microtome technique. It was found that the inner part of the nanoporous polyelectrolyte sphere was also efficiently filled with polyelectrolytes (FIG. 3b). At higher magnification (FIG. 3c) it can be observed that the porous structure was relatively homogeneous with a pore size of from 5 to 50 nm.

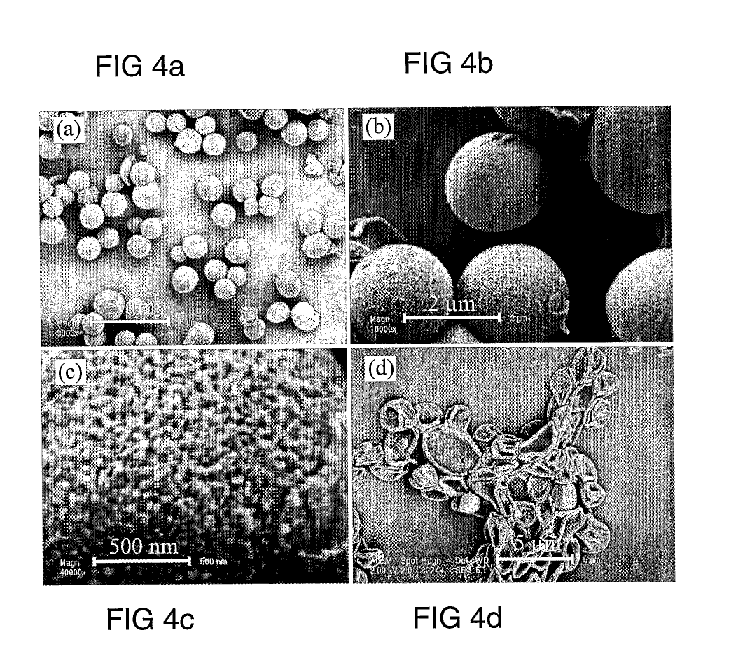

Scanning electron microscopy (SEM) was further used to observe the morphology of the particles (FIG. 4). No obvious aggregation of the particles is observed at low magnification (FIG. 4a). With the magnification increase, the roughness and porosity of the spheres becomes clear (FIG. 4b). At high magnification (FIG. 4c), abundant and homogeneous pores in the range of 10-40 nm were found.

FIG. 4d shows the collapsed capsule structure of the product prepared by the same procedure except without salt in the polyelectrolyte solution. This indicates that, in the presence of salt, polyelectrolyte can be highly coiled and able to penetrate into the mesopores of the templates.

The porosity and adsorption ability of the nanoporous polyelectrolyte sphere particles is also characterized through enzyme entrapment. Approximately 10 mg of the nanoporous polyelectrolyte spheres were dispersed in 15 mL of lysozyme (molecular weight 14.6 k Da) with a concentration of 1.0 mg mL⁻¹ in 50 mM phosphate buffer (pH 7.0) stock solution and shaken at room temperature for twenty fours. The enzyme retained by the particles was monitored by UV-vis spectroscopy, i.e., by monitoring the difference in solution between the protein absorbance at 280 nm before adsorption and after separating the supernatants via centrifugation at a speed of 1000 g for five minutes. For the NPS-5 particles, the weight will increase about 90% after lysozyme immobilization, which means nearly half the weight in the enzyme nanoporous polyelectrolyte sphere materials is contributed by the enzyme.

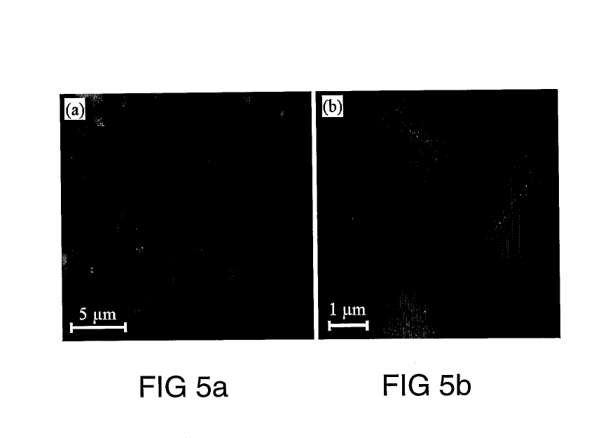

The immobilization and distribution of enzyme in the nanoporous polyelectrolyte sphere particles was further examined by confocal laser scanning microscopy (CLSM) of a cross section of individual particles. FIG. 5 shows the CLSM images of the NPS-5 spheres after incubating in fluorescein isothiocyanate-labelled lysozyme (FITC-lysozyme) for one hour, followed by washing with copious amounts of Milli-Q water. The bright spheres seen are due to the homogenous distribution of FITC-lysozyme in the nanoporous polyelectrolyte sphere particles with a relatively high enzyme amount (FIG. 5a). The fluorescence distribution in the nanoporous polyelectrolyte spheres is rather homogeneous, indicating effective immobilization of lysozyme molecule in the particles. Excellent enzyme immobilization ability of the nanoporous polyelectrolyte sphere particles is mostly caused by the abundant pores in nanoscale and high amount of functional chemical groups in the polyelectrolyte network.

The results demonstrate the success of layer-by-layer assembly of polyelectrolyte in mesoporous silica materials, to prepare nanoporous polyelectrolyte materials. The functional chemical group types and amounts can be controlled through layer-by-layer assembly. Compared with the traditional silane modification technology, significantly higher amounts of functional chemical groups (for example —NH₂, —COOH etc.) are expected to be grafted into the mesopores since the high molecular weight and long chain of polyelectrolyte molecules which may coil in the pores (previous silane modification is largely restricted to a single layer of functional group modification in the pore walls), hence influence the material adsorption properties and application.

Nanoporous polyelectrolyte materials were obtained after removal of the silicious skeleton. The size of the final nanoporous polyelectrolyte sphere particles can be controlled by adjusting the polyelectrolyte deposition layers. Excellent adsorption (e.g. enzyme immobilization) ability of the nanoporous polyelectrolyte spheres is found due to its abundant nanoscaled pore structures and high amount of functional groups in the polyelectrolyte networks. Electron microscopy data show that the nanoporous polyelectrolyte spheres of the invention have pores ranging from ca. 5-50 nm. The spheres show excellent capacity for immobilization of enzymes (lysozyme). Since the method is amenable to the deposition of diverse polyelectrolytes, the preparation of nanoporous polyelectrolyte spheres of controlled composition and functionality can be achieved by the present invention.

### Example 2

### Production of Nanoporous Protein Particles

### NPP with a Protein as One of the Polyelectrolyte Layers

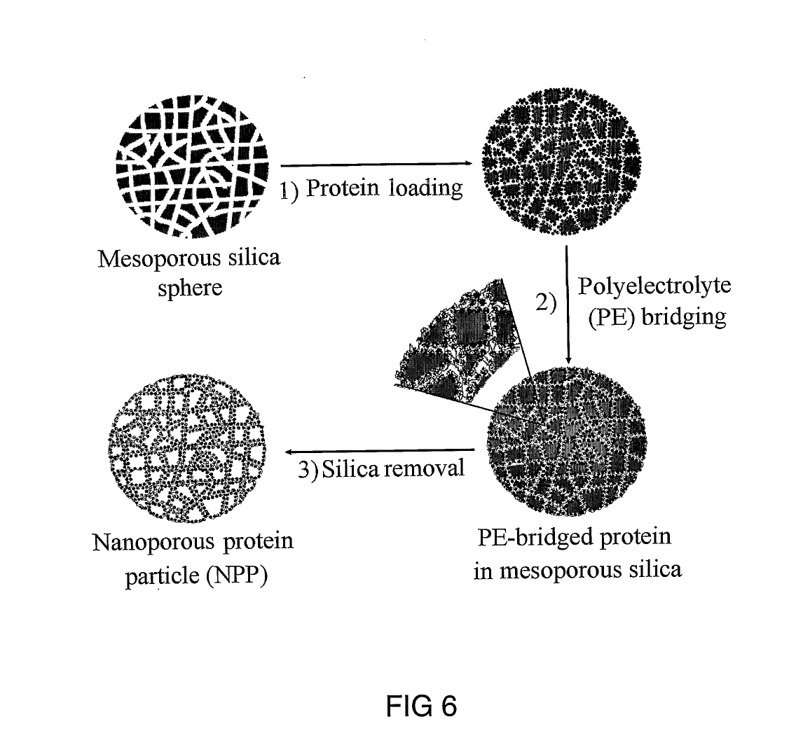

The general procedure was depicted in FIG. 6 and involves three main steps. The first involves immobilizing protein in the MS spheres by solution adsorption. Secondly, an oppositely charged polyelectrolyte (PE) is infiltrated into the protein-loaded mesopores, “bridging” the proteins. In the third step, the MS template is removed by exposure to a solution of hydrofluoric acid (HF)/ammonium fluoride (NH₄F), resulting in free-standing NPPs.

### Particle Production

Several proteins with different molecular weight, size and isoelectric point (pI) were chosen for investigation: lysozome (14.6 kDa, 3-4.5 nm, pI 11); cytochrome C (12 kDa, 3 nm, pI 10.3); and catalase (250 kDa, 10.4 nm, pI 5.4). The protein loading was performed by dispersing 10 mg of the MS particles in the protein solution (20 mL of 0.5 mg mL⁻¹ protein in 50 mM phosphate buffer (PB) at pH 7) and mixing at 20° C. for either 3 days (lysozyme or cytochrome C) or 7 days (catalase). The amount immobilized was determined by monitoring the difference in the protein absorbance in solution (lysozyme 280 nm; cytochrome C 530 nm; catalase 405 nm) before and after adsorption. The loadings for lysozyme, cytochrome C, and catalase are 400, 230, and 75 mg g⁻¹ MS, respectively.

Following several washing cycles to remove loosely adsorbed protein, the protein-loaded MS particles were dispersed in a 5 mg mL⁻¹ poly(acrylic acid) (PAA, M_{w} 8 000) solution at pH 4.5, which contained 0.1 M NaCl. PAA was allowed to infiltrate into the protein-loaded mesopores for 24 h at 20° C. Excess PAA was removed by two cycles of centrifugation and washing with 50 mM PB at pH 7. To enhance the stability of the protein/PAA layers, cross-linking of protein/PAA was performed by shaking the particles with 0.3 mL of EDC solution (60 mg mL⁻¹ in 50 mM PB) for 2 h at 20° C. The MS particle template was then dissolved by adding 2 mL of a 2 M HF/8 M NH₄F solution at 20° C. to the protein/PE loaded MS particles for 5 min, followed by two centrifugation (1500 g for 5 min)/water washing cycles. The resulting NPPs were stored in 50 mM PB at pH 7. The NPP materials composed of PE and lysozyme, cytochrome C, or catalase are herein denoted as NPP-lys, NPP-cyt, and NPP-cat, respectively.

### Nitrogen Adsorption Measurements

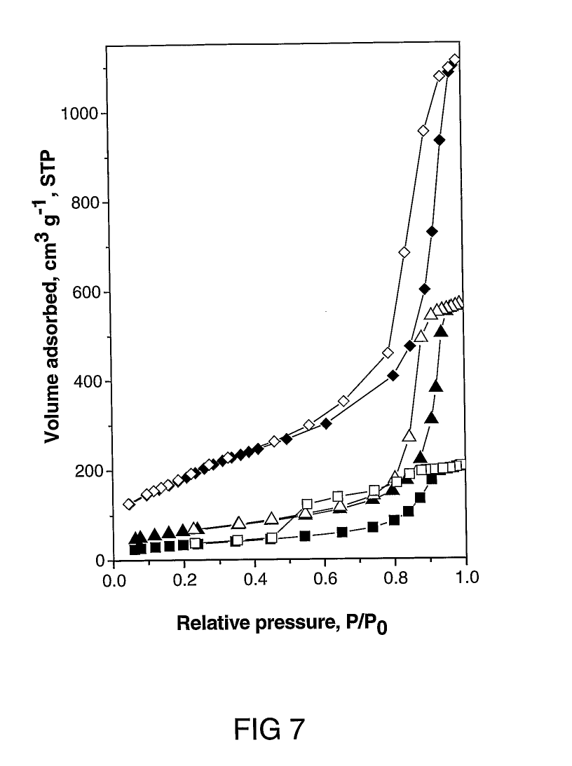

Nitrogen adsorption measurements were conducted to follow the variation in porosity of the MS spheres as a result of protein and PE infiltration. FIG. 7 shows the nitrogen isotherms for the MS spheres before and after lysozyme loading, and after PAA infiltration and cross-linking. The native MS has a surface area of 630 m²g⁻¹ and a pore volume of 1.72 cm³g⁻¹. After lysozyme immobilization, the surface area and pore volume of the particles significantly decreased to 230 m²g⁻¹ and 0.88 cm³g⁻¹, respectively, indicating high amount of protein is loaded in the mesopores. Thermogravimetric analysis (TGA) measurements showed the amount of lysozyme immobilized in the MS spheres was 41 wt %, which is in close agreement with the loading determined from UV-vis (40 wt %). The surface area and pore volume further decreased to 140 m²g⁻¹ and 0.40 cm³g⁻¹, respectively, after PAA infiltration. TGA experiments revealed that the amount of PAA infiltrated into the MS spheres was 7.5 wt %.

### Particle Stability

Structure stability was assessed by treatment of the lysozyme loaded MS spheres in solution with different concentration of (NH₄)₂SO₄. 1.5 mg of the lysozyme loaded particles was added to 1 mL of (NH₄)₂SO₄ solution with a salt concentration of 0, 0.1, and 0.5 M, respectively, and incubated for 150 min at 20° C. The mixture was then centrifuged and the protein content in the supernatant determined by UV-vis at 280 nm.

The stability of the PAA-connected catalase within the MS spheres was examined by dispersing 2 mg particles in 2 mL PB solution (50 mM at pH 7.0) at 5° C. for 14 days. The amount of protein desorbed during this time was determined by measuring the supernatant activity after centrifugation of the stock suspension.

### Results

PE bridging efficiency was assessed by treatment of the lysozyme loaded MS spheres in solution with different concentration of (NH₄)₂SO₄. About 9%, 25%, and 40% of the enzyme were desorbed from the lysozyme-loaded MS particles after the treatment with 0, 0.1, and 0.5 M (NH₄)₂SO₄, respectively. After cross-linking the protein with PAA by EDC, no enzyme was detected in the 0 and 0.1 M (NH₄)₂SO₄ solution, and only 0.18% lysozyme was desorbed in the 0.5 M (NH₄)₂SO₄ solution. The stability of the PAA-connected catalase within the MS spheres was examined by dispersing the samples in 50 mM PB solution at 5° C. for 14 days. The amount of protein desorbed during this time was determined by measuring the supernatant activity after centrifugation of the stock suspension. Catalase was selected because catalase decomposition of H₂O₂ is very sensitive, allowing the measurement of trace amounts of protein (one mole of catalase can decompose 5×10⁸ moles of H₂O₂ per min). Experiments showed that 10% of the adsorbed protein desorbed from the catalase-loaded MS particles during storage. After PAA infiltration, less than 7% of the immobilized protein desorbed. Protein loss was prevented by EDC cross-linking the protein/PAA-infiltrated MS spheres; negligible protein was detected in the supernatant. This indicates that the protein was firmly immobilized in the mesopores, and that the immobilization-bridging strategy provides an effective method for protein immobilization in porous materials. Further, the cross-linked PAA/catalase-loaded MS spheres showed no loss of activity over two weeks.

### Particle Characterization

Transmission electron microscopy (TEM) samples were prepared by placing a drop of a diluted capsule suspension (dispersed in water) onto a TEM grid. A Philips CM 120 microscope operated at 120 kV was used for analysis. A HP 8453 UV-vis spectrophotometer (Agilent, Palo Alto, Calif.) was used to monitor the enzyme loading, activity and release amount. Confocal laser scanning microscopy (CLSM) images were taken with an Olympus confocal system equipped with a 60× oil immersion objective. Adsorption-desorption measurements were conducted on a Micromeritics Tristar/surface area and porosity analyser at 77 K using nitrogen as the adsorption gas. The surface areas were calculated by the Brunauer-Emmett-Teller (BET) method. Thermogravimetric analysis (TGA) was performed on a Mettler Toledo/TGA/SDTA851e Module analyser.

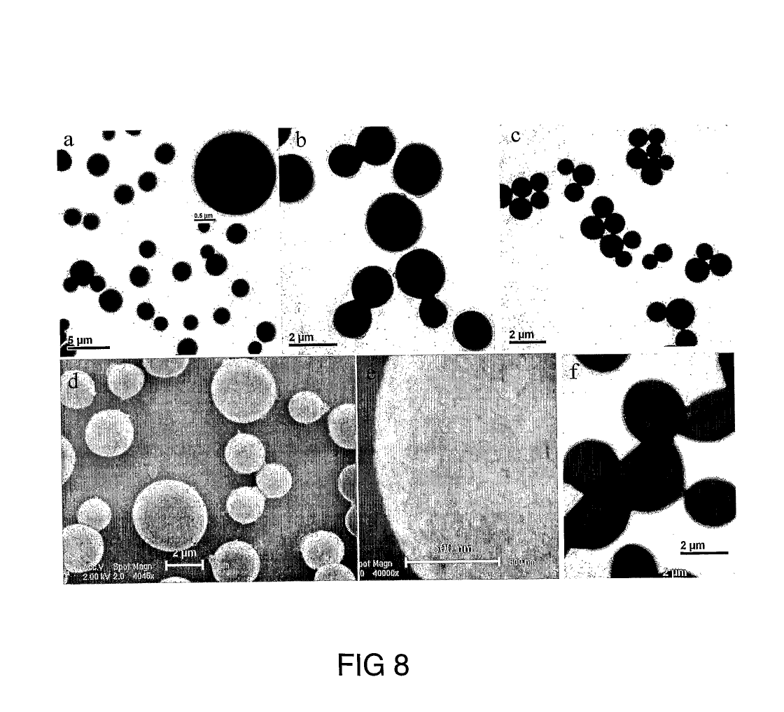

Transmission electron microscopy (TEM) was used to examine the NPPs obtained after silica removal. TEM shows that individual NPP-lys particles were produced, with no aggregation observed (FIG. 8a). These particles retained the original spherical shape of the MS templates, and did not show signs of collapse, as is typically observed for PE capsules. The NPP-lys had diameters ranging from 1.6-2.4 μm, some 20% smaller than the MS template particles (>90% of MS particles are within 2-3 μm). SEM also revealed the NPP-lys to be individual particles (FIG. 8d). At higher magnification, the surface roughness of the NPP-lys is apparent (FIG. 8e). The highly efficient template role of the MS spheres for the preparation of the NPPs is attributed to the disordered pore structure of the larger mesopores (10˜40 nm, 1.2 cm³g⁻¹) and the high surface area (630 m²g⁻¹) of the MS particles. NPPs were not formed if the PAA infiltration step was eliminated, even if EDC cross-linking was performed on the lysozyme-loaded MS spheres. This clearly indicates that PAA plays an essential role in connecting the proteins. When using cytochrome C, which has a similar size and pI as lysozome, the NPP-cyt formed (FIG. 2b) were similar in appearance to those shown in FIG. 8a. However, for catalase, which has a higher molecular weight and size (˜10.4 nm) and lower pI (5.4), considerably less protein loading was obtained in the MS spheres (˜7.5 wt %). Both particles similar to those shown in FIG. 8a-c as well as non-spherical (“collapsed”) particles (˜30%) were observed. The low protein loading most probably results in collapse of some of the particles upon drying (data not shown). This highlights the importance of high protein loadings in the MS spheres, which is largely determined by the protein size and the protein pI, to generate stable NPPs. Lowering the catalase deposition solution pH to ca. 5 did not yield higher loadings because of the complex interplay of electrostatic and secondary interactions associated with protein adsorption.

Several other PEs were used to bridge the lysozyme-loaded MS spheres to examine the effect of PE on the NPPs. When the polypeptide poly(L-glutamic acid) (PGA) was used and the PGA-lysozyme structure cross-linked with EDC, NPP-lys were also formed. These NPPs are similar in appearance to those obtained when PAA was used (FIG. 8a). In both the PAA/lysozyme and PGA/lysozyme systems, cross-linking was required to obtain spherical and intact NPPs. In contrast, stable NPP-lys were prepared without the cross-linking step if poly(sodium 4-styrenesulfonate) (PSS, M_{w} 70 000, 5 mg mL⁻¹ in 0.1 M NaCl) was used as the bridging PE (FIG. 8c). This can be explained by enhanced interactions between the lysozyme and PSS, compared with PAA or PGA. The NPP-lys prepared using PSS as the bridging PE had diameters ranging from 1.0-1.3 μm (FIG. 8c), about 50% smaller than the MS template particles. This shrinkage may be caused by the higher mobility of the non-cross-linked protein. These results indicate that various PEs can be used as bridging polymers and that the PE type can determine the final size of the NPPs. The PE would also likely govern the porosity and stability of the protein spheres.

Experiments were conducted using linker PEs with different molecular weights to provide evidence for the infiltration of PAA and subsequent formation of connected protein layers in the mesopores. The use of PAA with a much high molecular weight (250 000 Da) than that used to generate the NPP-lys shown in FIG. 8a-c (70 000 Da), resulted in “collapsed capsule” structures (FIG. 8f). These data suggest that the high molecular weight PAA is too large to enter the protein-loaded mesopores, and therefore is mainly restricted to the outside surface of the particles. Hence, only a capsule-like complex of lysozyme and PAA was formed. The presence of salt in the PE solution, which affects the molecular size, also plays an essential role in the preparation of NPPs. Only capsule-like materials (similar to those shown in FIG. 8d) were obtained when PSS (70 000 Da) solutions without salt were used. This is in stark contrast to the NPP-lys prepared when salt (0.1 M NaCl) was used in the PSS adsorption solution (see FIG. 8c). In the absence of salt, PSS adopts a more linear conformation, and is therefore also restricted to adsorption mainly on the outside particle surface.

### Confocal Laser Scanning Microscopy

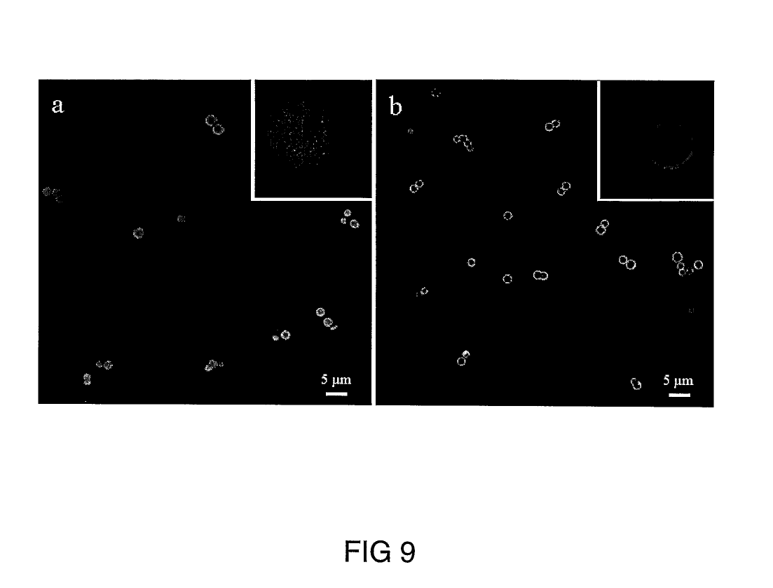

Confocal laser scanning microscopy (CLSM) experiments were conducted to investigate the inner structure of the NPPs. FIG. 9 shows CLSM images of the NPP-lys after incubation in Rhodamine 6G (M_{w} 479 Da) solution for 60 min, followed by washing with water. The bright spheres seen are due to the homogeneous distribution of the dye in the particles (FIG. 9a inset), reflecting the non-hollow structure and the porous nature of the particles. This suggests that NPPs can be used as bioreactors and as drug loading vehicles. The CLSM image also shows that NPPs are well separated in solution. For the proteins linked by PAA of high molecular weight (250 000 Da), distinct fluorescent rings are observed, indicating localization of most of the dye on the outside surface (FIG. 9b). In this case, a hollow-structured lysozyme-PAA layer is formed, which is in accordance with the capsule structure observed from TEM (FIG. 8d).

The NPP-lys has a lysozyme: PAA composition of ˜5:1 (weight to weight), that is, about 83 wt % of the particles are protein. The protein content is significantly higher than that of the proteins adsorbed in preformed nanoporous PAA/PAH spheres (ca. 1:1) and in the MS spheres (ca. 0.4:1). Although PE is used to bridge the protein molecules, the NPPs are composed mostly of protein: the biomolecule content (mass:mass) is 12.5 times higher than protein loaded in MS spheres. Further the NPPs composed entirely of biocompatible materials can be prepared by using, for example, polypeptides (i.e. PGA) as the bridging molecule. The high protein content of such particles is of interest in drug delivery, especially for improving drug efficacy and decreasing side effects.

### Enzyme Activity Assay

The enzyme activity was determined spectrophotometrically using H₂O₂ as a substrate. The NPP-cat (or free enzyme) was added to 11 mM H₂O₂ in 50 mM PB solution (pH 7.0) with rapid stirring. The decrease in absorbance at 240 nm (with an extinction coefficient of 0.041 mmol⁻¹ cm⁻¹) with time was recorded immediately after the enzyme was mixed into the above solution at 20° C. One unit of catalase will decompose 1 μmol of H₂O₂ per minute at pH 7.0 and 20° C.

The activity of catalase-loaded in the MS particles (˜66% of the free protein) is normalized as 100%. The activity slightly decreased to ˜91% after PAA infiltration. After EDC cross-linking, the MS-immobilized and cross-linked protein retains 75% of its activity (relative to catalase-loaded MS particles), which is significantly higher than that for catalase immobilized in MS spheres and encapsulated with four PDDA/PSS bilayers (ca. 50%), and for catalase cross-linked by glutaraldehyde in chitosan beads (˜1% activity). After silica removal, the catalase activity increased from 75% to 86% (corresponding to 57% of the free protein in bulk solution), suggesting increased substrate accessibility after removal of the MS template.

### Example 3

### Formation of Fibrous Particles

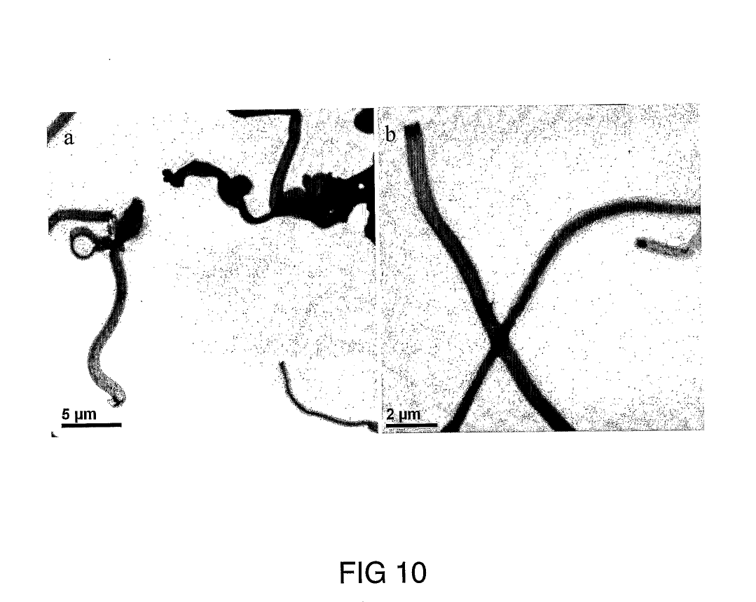

The method is also amendable to prepare nanoporous protein structures with various morphologies. Nanoporous protein fibers (NPF) were prepared by using MS templates with fiber morphologies using the procedures outlined in examples 1 and 2. MS fibers have similar porosity to the MS spheres used, with the exception of the worm-shaped morphology about 1 μm in diameter, and are 10 to 30 μm in length. FIG. 10 shows the NPF at different magnifications after silica removal. The protein fibers have lengths of tens of micrometers and diameters of hundreds of nanometers, closely mimicking the sizes of the MS fibers. Realization of these structures indicates that the PE-bridging template synthesis provides a facile method to control protein morphologies.

### INDUSTRIAL APPLICABILITY

Layer-by-layer coating for nanoporous polyelectrolyte spheres may be used in many applications, for example, in drug delivery with selectivity due to the control of surface functionality. The nanoporous polyelectrolyte particles have high adsorption capacity and can be used in a number of applications, for example drug delivery, separation of biomaterials such as enzymes or non-bio materials such as separation of heavy metal ions or toxic organics molecules, used as adsorbents for dyes and may also be useful in the controlled release of fragrances in certain applications.

The nanoporous protein particles can be prepared with protein contents as high as 83 wt % and from a range of polyelectrolytes/proteins, including biocompatible bridging polymers such as polypeptides. The well retained protein activities make such particles promising for functional protein drug delivery

One particularly attractive use of the materials of the invention is in methods of delivering an active agent to a target site the method including the steps of (I) adsorbing the active agent onto a multilayer polyelectrolyte material of the invention and (ii) delivering the polyelectrolyte material to the target site. The active agent may be adsorbed in any of a number of ways but us typically adsorbed by suspending a polyelectrolyte material of the invention into a solution of the active agent. The active agent is adsorbed onto the polyelectrolyte material which can then be isolated from the solution. The polyelectrolyte material with eth active agent adsorbed thereon may then be delivered to the target site such as by administration to the site. Any suitable active agent may be chosen such as therapeutic agent including pharmaceuticals, veterinary chemicals and the like. Alternatively the active agent may be a fragrance or a cleaning chemical which is intended to be delivered to its site of action.

The polyelectrolyte material of the invention also finds use as a micro reactor. It is found that the materials adsorb compounds and can thus be used to adsorb one or more reactive species allowing them to be held proximal to each other to facilitate reaction.

Accordingly another application of the materials is in methods of conducting a chemical reaction including contacting a solution containing one or more reactants with a polyelectrolyte material of the invention. The step of contacting preferably involves addition of the polyelectrolyte material of the invention to a solution containing the reactant(s) in question. The chemical reaction may be carried out by the polyelectrolyte material acting as a micro reactor for the chemical reactant(s) as discussed above or it may actually take part in the reaction. In a particularly preferred embodiment the reaction is an enzymatic reaction, preferably an enzymatic catalytic reaction of a reactant. In a most preferred embodiment the polyelectrolyte material catalyses the reaction.

As a result of their ability to adsorb chemical compounds the polyelectrolyte materials of the invention may be used as adsorbents. Accordingly another use is in methods of removing a compound from solution including contacting the solution with a polyelectrolyte material of the invention, allowing sufficient time for the compound to be adsorbed by the polyelectrolyte material and removing the polyelectrolyte material from the solution. This method may be used to isolate drugs from solution or in the purification of solutions containing trace amounts of compounds that it is desired be removed from solution.

Finally, it is to be understood that various alterations, modifications and/or additions may be introduced into the constructions and arrangements of parts previously described without departing from the spirit or ambit of the invention.

1. A porous multilayer polyelectrolyte material including at least two layers of polyelectrolyte material.

2. A porous multilayer polyelectrolyte material according to claim 1 wherein the material includes at least two layers of oppositely charged polyelectrolyte material.

3. A material according to claim 1 or 2 wherein the material includes pores with a pore size of from 5 to 50 nm.

4. A material according to any one of claims 1 to 3 wherein the material includes pores with a pore size of from 10 to 50 nm.

5. A material according to claim 3 or 4 wherein the pores are interconnecting to produce an interconnected porous network.

6. A material according to any one of claims 1 to 5 wherein the material includes from two to ten layers of polyelectrolyte material.

7. A material according to any one of claims 1 to 6 wherein the material includes from two to eight layers of polyelectrolyte material.

8. A material according to any one of claims 1 to 7 wherein the material includes two layers of polyelectrolyte material.

9. A material according to any one of claims 1 to 8 wherein each layer of polyelectrolyte material is oppositely charged to the layer(s) of polyelectrolyte material adjacent to it.

10. A material according to any one of claims 1 to 9 wherein the material includes at least two adjacent layers of polyelectrolyte material with the same charge.

11. A material according to any one of claims 1 to 10 wherein one or more of the layers of polyelectrolyte material is cross-linked to an adjacent layer.

12. A material according to any one of claims 1 to 11 wherein one or more of the layers of polyelectrolyte material is internally cross linked.

13. A material according to any one of claims 1 to 8 wherein each layer includes a polyelectrolyte material independently selected from the group consisting of polymers, biodegradable polymers, poly(amino acids), peptides, glycopeptides, polypeptides. peptidoglycans, glycosaminoglycans, glycolipids, lipopolysaccharides, proteins, glycoproteins, polycarbohydrates; polynucleotides, modified biopolymers; polysilanes, polysilanols, polyphosphazenes, polysulfazenes, polysulfide, polyphosphates, nucleic acid polymers, nucleotides, polynucleotides, RNA and DNA.

14. A material according to any one of the preceding claims wherein each layer includes a polyelectrolyte material independently selected from the group consisting of polyglycolic acid (PGA), polylactic acid (PLA), poly-2-hydroxy butyrate (PHB), gelatins, (A, B) polycaprolactone (PCL), poly(lactic-co-glycolic acid) (PLGA), carboxymethyl cellulose, carboxymethyl dextran, poly(allylamine hydrochloride), poly(acrylic acid), poly(sodium 4-styrene sulphonate), poly (diallyidimethylammonium chloride), poly(vinylsulfate), poly(L-glutamic acid) and poly(L-lysine) or a mixture thereof.

15. A material according to any one of claims 1 to 14 wherein each polyelectrolyte material has a molecular weight of at least 100.

16. A material according to any one of claims 1 to 15 wherein each polyelectrolyte material has a molecular weight of 100 to 1,000,000.

17. A material according to any one of claims 1 to 16 wherein each polyelectrolyte material has a molecular weight of from 500, to 500,000.

18. A material according to any one of claims 1 to 17 wherein each polyelectrolyte material has a molecular weight of from 500 to 100,000.

19. A material according to any one of claims 1 to 18 wherein each polyelectrolyte material has a molecular weight of from 1000 to 100,000.

20. A material according to any one of claims 1 to 19 wherein the polyelectrolyte material in at least one layer contains an amine group.

21. A material according to any one of claims 1 to 20 wherein the polyelectrolyte material in at least one layer contains a carboxylic group.

22. A material according to any one of claims 1 to 21 wherein the material includes at least one layer of poly(acrylic acid).

23. A material according to any one of claims 1 to 22 wherein the material includes at least one layer of poly(allylamine hydrochloride).

24. A material according to any one of claims 1 to 15 wherein the material in at least one polyelectrolyte layer is selected from the group consisting of peptides, glycopeptides, polypeptides. peptidoglycans, glycosaminoglycans, glycolipids, lipopolysaccharides, proteins, glycoproteins and polynucleotides.

25. A material according to any one of claims 1 to 24 wherein at least one polyelectrolyte layer is a protein layer.

26. A material according to claim 25 wherein the protein has a molecular weight of from 1 to 500 kDa.

27. A material according to claim 25 wherein the protein is selected from the group consisting of lysosome, cytochrome C, catalase, bovine serum albumin, immunoglobulin G, protease, RNase A, trypsin, conalbumin, lactoglobulin, myoglobin, ovalbumin, papain, penicillin acylase, and subtilisin Carlsberg.

28. A material according to any one of claims 1 to 27 wherein the porous multilayer polyelectrolyte material is spherical or substantially spherical.

29. A porous polyelectrolyte material according to any one of the preceding claims wherein the material is self-supporting.

30. A method of manufacturing a porous multilayer polyelectrolyte material including the steps of:
(vii) providing a porous template;
(viii) depositing layer-by-layer polyelectrolyte material onto the porous template; and
(ix) removing the template by exposure to a suitable solvent.

31. A method according to claim 30 wherein the template has an interconnected network of pores.

32. A method according to claim 31 wherein the template includes pores with a pore size in the range 2 to 50 nm.

33. A method according to any one of claims 30 to 32 wherein the template is a silica template.

34. A method according to any one of claims 30 to 33 wherein the template is selected from the group consisting of planar supports, powder particles, fibres, films, membranes and spheres.

35. A method according to any one of claims 30 to 34 wherein the template is spherical or substantially spherical.

36. A method according to any one of claims 30 to 35 wherein the exposed surface of the template has been modified.

37. A method according to claim 36 wherein the exposed surface has been modified by grafting 3-aminopropyltriethoxysilane (APTS) onto the exposed surface.

38. A method according to any one of claims 30 to 37 wherein the polyelectrolyte material is deposited in layers of alternating charge.

39. A method according to any one of claims 30 to 38 wherein each layer is deposited by contacting the template with a solution containing the polyelectrolyte material to be deposited.

40. A method according to claim 39 wherein the solution has a concentration of polyelectrolyte material of 0.001 to 100 mg mL⁻¹.

41. A method according to claim 39 or 40 wherein the solution has a concentration of polyelectrolyte material of 0.1 to 30 mg mL⁻¹.

42. A method according to any one of claims 39 to 41 wherein the solution has a concentration of polyelectrolyte material of 0.5 to 10 mg mL⁻¹.

43. A method according to any one of claims 39 to 42 wherein the solution includes a salt.

44. A method according to claim 43 wherein the salt has a concentration of from 0.001 to 5 M.

45. A method according to claim 43 or 44 wherein the salt has a concentration of from 0.05 to 5 M.

46. A method according to any one of claims 43 to 45 wherein the salt has a concentration of from 0.1 to 1 M.

47. A method according to any one of claims 43 to 46 wherein the salt is sodium chloride.

48. A method according to any one of claims 39 to 47 wherein the contacting is carried out for from 15 minutes to 24 hours.

49. A method according to any one of claims 39 to 48 wherein the contacting is carried out for from 2 hours to 20 hours.

50. A method according to any one of claims 39 to 49 wherein the contacting is carried out for from 4 hours to 12 hours.

51. A method according to any one of claims 39 to 50 wherein during contacting the solution is subjected to ultrasound irradiation.

52. A method according to any one of claims 30 to 51 wherein each layer of polyelectrolyte material is cross-linked after being deposited and before deposition of a further layer.

53. A method according to claim 52 wherein the polyelectrolyte layer is cross-linked by heating at a temperature of from 100° C. to 250° C.

54. A method according to claim 52 wherein the polyelectrolyte layer is cross-linked using 1-ethyl-3-(3-dimethylaminopropyl)carbodiimide hydrochloride.

55. A method according to any one of claims 30 to 54 wherein a plurality of layers are deposited.

56. A method according to any one of claims 30 to 55 wherein from two to ten layers are deposited.

57. A method according to claim 50 or 51 wherein two to eight layers are deposited.

58. A method according to any one of claims 30 to 57 wherein the polyelectrolyte material deposited to form each layer is independently selected from the group consisting of polymers, biodegradable polymers, poly(amino acids), peptides, glycopeptides, polypeptides. peptidoglycans, glycosaminoglycans, glycolipids, lipopolysaccharides, proteins, glycoproteins, polycarbohydrates; polynucleotides, modified biopolymers; polysilanes, polysilanols, polyphosphazenes, polysulfazenes, polysulfide, polyphosphates, nucleic acid polymers, nucleotides, polynucleotides, RNA and DNA.

59. A method according to any one of claims 30 to 58 wherein the polyelectrolyte material deposited to form each layer is independently selected from the group consisting of polyglycolic acid (PGA), polylactic acid (PLA), poly-2-hydroxy butyrate (PHB), gelatins, (A, B) polycaprolactone (PCL), poly(lactic-co-glycolic acid) (PLGA), carboxymethyl cellulose, carboxymethyl dextran, poly(allylamine hydrochloride), poly(acrylic acid), poly(sodium 4-styrene sulphonate), poly(diallyldimethylammonium chloride), poly(vinylsulfate), poly(L-glutamic acid) and poly(L-lysine).

60. A method according to any one of claims 30 to 59 wherein each polyelectrolyte material has a molecular weight of at least 100.

61. A method according to any one of claims 30 to 60 wherein each polyelectrolyte material has a molecular weight of 100 to 1,000,000.

62. A method according to any one of claims 30 to 61 wherein each polyelectrolyte material has a molecular weight of from 500, to 500,000.

63. A method according to any one of claims 30 to 62 wherein each polyelectrolyte material has a molecular weight of from 500 to 100,000.

64. A method according to any one of claims 30 to 63 wherein each polyelectrolyte material has a molecular weight of from 1000 to 100,000.

65. A method according to any one of claims 30 to 64 wherein the polyelectrolyte material deposited to form at least one layer contains an amine group.

66. A method according to any one of claims 30 to 65 wherein the polyelectrolyte material deposited to form at least one layer contains a carboxylic group.

67. A method according to any one of claims 30 to 66 wherein the polyelectrolyte material deposited to form at least one layer is poly(acrylic acid).

68. A method according to any one of claims 30 to 67 wherein the polyelectrolyte material deposited to form at least one layer is poly(allylamine hydrochloride).

69. A method according to any one of claims 30 to 68 wherein the polyelectrolyte material deposited to form at least one layer is selected from the group consisting of poly(amino acids), peptides, glycopeptides, polypeptides. peptidoglycans, glycosaminoglycans, glycolipids, lipopolysaccharides, proteins, glycoproteins, polycarbohydrates; polynucleotides, nucleic acid polymers, nucleotides, polynucleotides, RNA and DNA.

70. A method according to any one of claims 30 to 69 wherein the polyelectrolyte material deposited to form at least one layer is a protein.

71. A method according to claim 70 wherein the protein has a molecular weight of from 1 to 500 kDa.

72. A method according to claim 70 wherein the protein is selected from the group consisting of lysosome, cytochrome C, catalase, bovine serum albumin, immunoglobulin G, protease, RNase A, trypsin, conalbumin, lactoglobulin, myoglobin, ovalbumin, papain, penicillin acylase, and subtilisin Carlsberg.

73. A method according to any one of claims 30 to 72 wherein the removal of the template involves exposure to hydrofluoric acid.

74. A method according to claim 73 wherein the hydrofluoric acid has a concentration of from 0.01 to 10 M.

75. A method of delivering an active agent to a target site the method including the steps of (I) adsorbing the active agent onto a multilayer polyelectrolyte material according to any one of claims 1 to 29 and (ii) delivering the polyelectrolyte material to the target site.

76. A method according to claim 75 wherein the active agent is a pharmaceutical.

77. Use of a polyelectrolyte material according to any one of claims 1 to 29 as a micro reactor.

78. A method of conducting a chemical reaction including contacting a solution containing one or more reactants with a polyelectrolyte material according to any one of claims 1 to 29.

79. A method according to claim 78 wherein the reaction is an enzymatic reaction.

80. A method according to claim 79 wherein the enzymatic reaction is the enzymatic catalytic reaction of a reactant.

81. A method according to claim 80 wherein the polyelectrolyte material catalyses the reaction.

82. A method of removing a compound from solution including contacting the solution with a polyelectrolyte material according to any one of claims 1 to 29, allowing sufficient time for the compound to be adsorbed by the polyelectrolyte material and removing the polyelectrolyte material from the solution.
